(19) United States
(12) Patent Application Publication
(10) Pub. No.: US 2011/0150777 A1
(43) Pub. Date: Jun. 23, 2011

(54) Magnetic resonance tomography imaging

(76) Inventor: Sebastian Schmidt, Weisendorf (DE)

(21) Appl. No.: 12/968,310

(22) Filed: Dec. 15, 2010

(30) Foreign Application Priority Data
Dec. 18, 2009 (DE) 102009054956.0

(51) Int. Cl. A61K 49/06; C07F 19/00; C07C 19/08; C08B 11/02; C08B 31/10; A61P 43/00; C22C 38/00

(52) U.S. Cl. 424/9.3; 534/16; 570/134; 536/99; 536/111; 420/8

(57) ABSTRACT

A medical magnetic resonance tomography apparatus and a contrast agent for magnetic resonance tomography imaging of cavities in a patient are provided. The contrast agent is formed from a highly viscous magnetic resonance contrast agent. The highly viscous magnetic resonance contrast agent includes a substance. A specific magnetic resonance contrast can be generated by the substance.

### CROSS REFERENCE TO RELATED APPLICATIONS

This application claims priority of German application No. 10 2009 054 956.0 filed Dec. 18, 2009, which is incorporated by reference herein in its entirety.

### FIELD OF THE INVENTION

The invention relates to magnetic resonance tomography imaging.

### BACKGROUND OF THE INVENTION

In the magnetic resonance tomography imaging of cavities in patients, it is often necessary to at least partially fill the cavity with a contrast agent, in order to achieve a high contrast when displaying the inner wall of the cavity. However magnetic resonance tomography imaging of a uterus has proven problematical since the viscosity of conventional magnetic resonance contrast agents is too low and they immediately escape again through the cervix.

Furthermore, a highly viscous contrast agent is known from EP 1 793 860, which is mainly used for an ultrasound imaging. The contrast agent is only inadequate for a magnetic resonance imaging, since it only produces a poor contrast here.

### SUMMARY OF THE INVENTION

The object underlying the present invention is in particular to provide a contrast agent for a magnetic resonance tomography imaging, which has a high viscosity and a high contrast characteristic for a magnetic resonance tomography imaging. The object is achieved by the features of the claims.

The invention is based on a magnetic resonance tomography imaging of cavities in a patient with a medical magnetic resonance tomography apparatus and a contrast agent.

It is proposed that the contrast agent is formed from a highly viscous magnetic resonance contrast agent, which includes a substance by means of which a specific magnetic resonance contrast can be generated. In this context, the term cavity of a patient is in particular to be understood as a cavity in the gastrointestinal region and/or the uterus etc. Furthermore, the term highly viscous magnetic resonance contrast agent is understood to mean in particular a viscous contrast agent, which generates a specific magnetic resonance contrast during magnetic resonance tomography imaging. A viscosity of the highly viscous magnetic resonance contrast agent of greater than 1000 mPa·s, advantageously greater than 1500 mPa·s and particularly preferably greater than 2000 mPa·s is preferred. The term substance is herewith intended to mean in particular an ingredient and/or a mixture of substances and/or at least one component of the highly viscous magnetic resonance contrast agent. The inventive embodiment enables a cavity, in particular the uterus, advantageously to be examined with a high contrast in terms of imaging by means of the magnetic resonance contrast agent together with the magnetic resonance tomography apparatus.

Furthermore, it is proposed that the highly viscous magnetic resonance contrast agent has a viscosity between 2000 mPa·s and 4000 mPa·s, as a result of which an escape, in particular a leakage out of the uterus, of the highly viscous magnetic resonance contrast agent prior to a magnetic resonance tomography measurement can be prevented. The highly viscous magnetic resonance contrast agent preferably has a viscosity between 2500 mPa·s and 4000 mPa·s and particularly preferably a viscosity between 3000 mPa·s and 4000 mPa·s.

It is also proposed for the highly viscous magnetic resonance contrast agent to have a pH value between 5.5 and 7.5 and particularly preferably a pH value between 5.5 and 6. In addition to a good contrast characteristic, a good tolerance of the contrast agent on account of the slightly acidic pH value can be achieved for a patient. In particular, a good tolerance can be achieved here when the contrast agent is used for a uterus examination by means of the magnetic resonance tomography apparatus.

The highly viscous magnetic resonance contrast agent preferably includes at least one highly viscous base material, like for instance a cellulose derivative, e.g. methyl cellulose, and/or a starch derivative, e.g. hydroxyethyl starch.

In addition to the highly viscous base material, the highly viscous magnetic resonance contrast agent can also include a buffer solution and/or a buffer mixture, like for instance an acetic acid acetate buffer, a citric acid phosphate buffer, a citrate-citrate buffer etc. The buffer solution and/or the buffer mixture in particular has the task of keeping the pH value of the highly viscous magnetic resonance contrast agent essentially within a buffer region, in particular within a region with a pH value between 5.5 and 7.5 and particularly preferably with a pH value between 5.5 and 6.0 constant and buffering fluctuations in the pH value.

The substance of the highly viscous magnetic resonance contrast agent which generates the specific magnetic resonance contrast is formed in a further embodiment of the invention preferably by a dissolved substance in a solution, in particular in the highly viscous base material of the highly viscous magnetic resonance contrast agent, and/or by a solution. The substance here is particularly advantageously at least partially formed from a gadolinium chelate, like for instance gadolinium diethylenetriaminepentaacetic acid (Gd-DTPA), and/or at least partially by a fluorine compound, like for instance a perfluorinated carbon compound. Furthermore, the substance can also be formed at least partially by a hyperpolarized substance, so that a particularly strong contrast can be achieved with the magnetic resonance tomography imaging. Furthermore, in an alternative embodiment of the invention, the substance producing the specific magnetic resonance contrast can also be formed at least partially by compounds including elements and/or atomic nuclei, which can be verified and/or mapped by means of the magnetic resonance tomography imaging, like for instance ¹³C and/or ¹⁵N etc.

In an advantageous development of the invention, it is proposed that the substance of the highly viscous magnetic resonance contrast agent which generates the specific magnetic resonance contrast is formed at least partially from iron particles. A positive iron contrast can be achieved here in particular during magnetic resonance tomography imaging. The iron particles preferably comprise a particle size with a diameter of approximately 5 nm to approximately 500 nm.

Furthermore, the invention is based on a magnetic resonance contrast agent for a medical imaging of cavities with a magnetic resonance tomography apparatus.

It is proposed that the magnetic resonance contrast agent has a highly viscous base material and a substance, by means of which a specific magnetic resonance contrast can be generated. A cavity, in particular the uterus, can advantageously be examined with a high contrast during imaging by means of the magnetic resonance contrast agent together with the magnetic resonance tomography apparatus.

### BRIEF DESCRIPTION OF THE DRAWINGS

Further advantages result from the following description of the drawings. Exemplary embodiments of the invention are shown in the drawing. The drawings, the description and the claims contain a combination of numerous features. Expediently the person skilled in the art will also consider the features individually and combine them to form meaningful further combinations.

In which:

FIG. 1 shows a flow diagram of a magnetic resonance tomography imaging process,

FIG. 2 shows a schematic representation of a magnetic resonance tomography apparatus,

FIG. 3 shows a highly viscous base material of a magnetic resonance contrast agent which is formed from methyl cellulose,

FIG. 4 shows a highly viscous base material of a magnetic resonance contrast agent which is formed from hydroxyethyl starch,

FIG. 5 shows a contrast-generating material of the magnetic resonance contrast agent formed from a gadolinium chelate, and

FIG. 6 shows a contrast-generating material of the magnetic resonance contrast agent which is formed from a perfluorinated carbon.

### DETAILED DESCRIPTION OF THE INVENTION

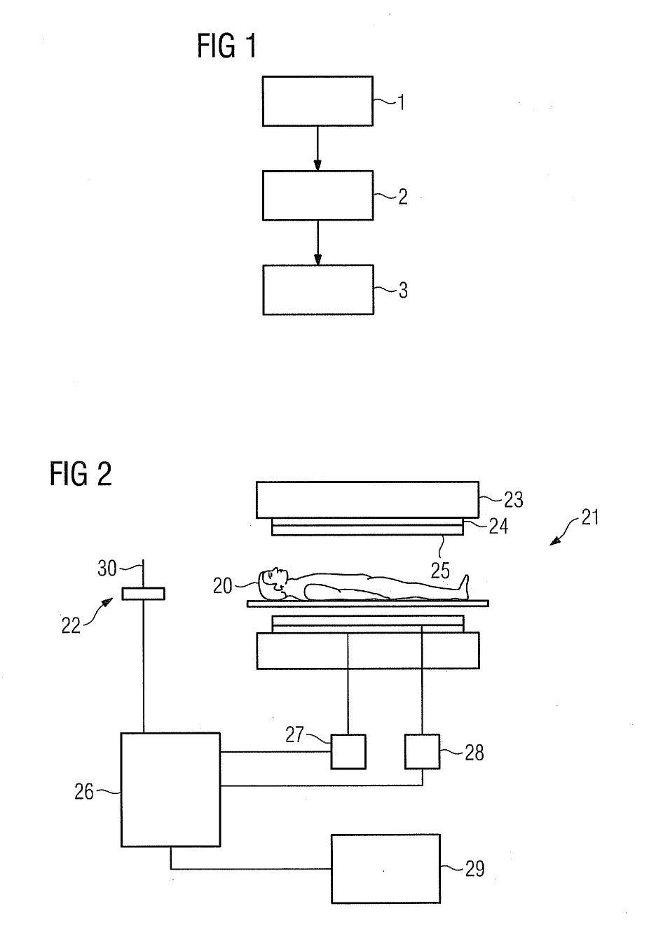

FIG. 1 shows a diagram of a magnetic resonance tomography imaging process for an imaging of cavities, in particular of the uterus, in patients 20 by means of a magnetic resonance tomography apparatus 21. During operation of the magnetic resonance tomography apparatus 21, a contrast agent is initially added in a contrast agent adding step 1 by means of a contrast agent unit 22 of the magnetic resonance tomography apparatus 21, said contrast agent being formed from a highly viscous magnetic resonance contrast agent. The contrast agent unit 22 to this end comprises an insertion element 30 formed from an injection needle, by way of which the contrast agent is added. The contrast agent can be added here manually by an operator of the magnetic resonance tomography apparatus 21 or automatically by means of the magnetic resonance tomography apparatus 21, with the contrast agent unit 22 being controlled for this process by way of a control unit 26 of the magnetic resonance tomography apparatus 22 (FIG. 2).

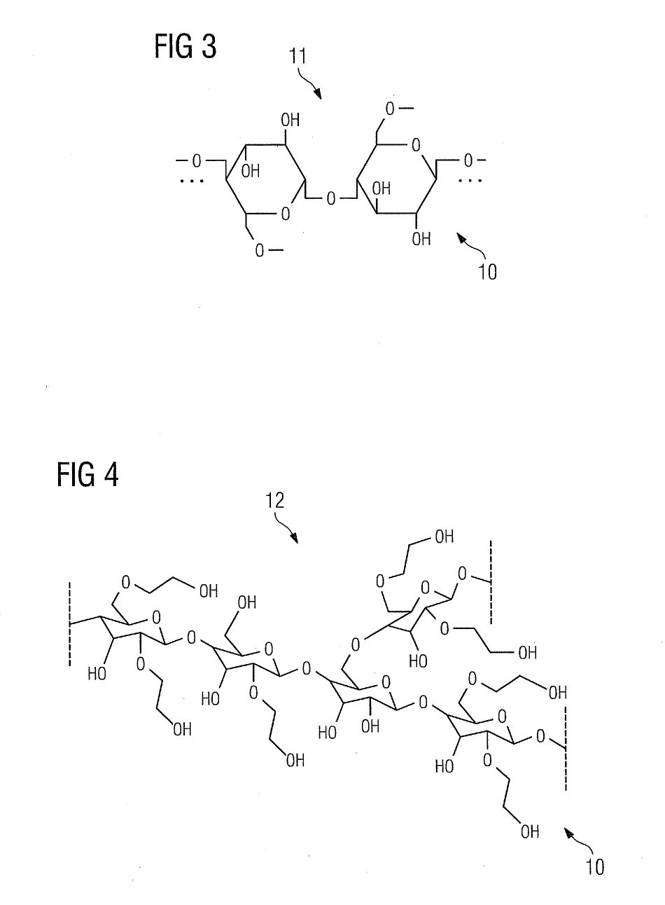

The highly viscous magnetic resonance contrast agent includes a highly viscous base material 10, which is formed for instance from a cellulose derivative, e.g. methyl cellulose 11 (FIG. 3), and/or from a starch derivative, e.g. hydroxyethyl starch (FIG. 4). The highly viscous base material 10 achieves a viscosity between 2000 mPa·s and 4000 mPa·s, advantageously between 2500 mPa·s and 4000 mPa·s and particularly preferably between 3000 mPa·s and 4000 mPa·s of the highly viscous magnetic resonance contrast agent. The highly viscous base material 10 achieves a highly viscous feature of the magnetic resonance contrast agent, which at least reduces and/or prevents an unwanted escape and/or leakage of the magnetic resonance contrast agent out of the cavity, in particular the uterus.

Furthermore, the highly viscous magnetic resonance contrast agent comprises a pH value between 5.5 and 7.5, and particularly preferably a pH value between 5.5 and 6.0, so that a good tolerance particularly in the region of the vagina and the uterus can be achieved for a patient 20. To stabilize the pH value, the highly viscous magnetic resonance contrast agent also includes a buffer solution, which is formed for instance from an acetic acid acetate buffer solution and/or a citric acid phosphate buffer solution and/or a citrate-citrate buffer solution etc.

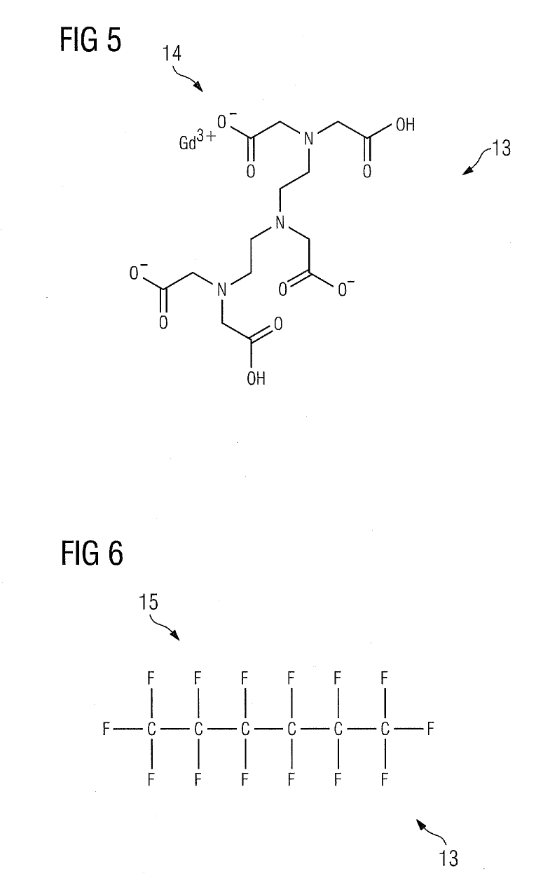

The highly viscous magnetic resonance contrast agent also comprises a substance 13 generating a contrast in order to generate a specific magnetic resonance contrast with a magnetic resonance tomography imaging. The substance 13 generating the specific magnetic resonance contrast is preferably formed from a substance and/or solution dissolved in a solvent. The substance 13 can be formed here from a gadolinium chelate, like for instance gadolinium diethylenetriaminepentaacetic acid 14 (Gd-DTPA) (FIG. 5), and/or at least partially by a fluorine compound, like for instance a perfluorinated carbon compound 15 (FIG. 6). Furthermore, the substance 13 can also be formed at least partially from a hyperpolarized substance 13.

Furthermore, the substance 13 producing the specific magnetic resonance contrast can also be formed at least partially by compounds including elements and/or atomic nuclei, which can be verified and/or mapped by means of the magnetic resonance tomography imaging, like for instance ¹³C and/or ¹⁵N etc. The iron particles preferably comprise a particle size with a diameter of approximately 5 nm to approximately 500 nm.

After the contrast agent adding step 1, a magnetic resonance measurement 2 is undertaken by means of the magnetic resonance tomography apparatus 21, which has magnets 23 for generating a strong and constant magnetic field. Furthermore, the magnetic resonance tomography apparatus 21 comprises gradient coils 24, which are provided to generate a linear gradient field, and high frequency coils 25. The gradient coils 24 are controlled by the control unit 26 by way of a gradient amplifier 27. The high frequency coils 25 are controlled by the control unit 26 by way of a high frequency amplifier 28. Furthermore, the magnetic resonance tomography apparatus 21 comprises a screen display 29, by means of which magnetic resonance recordings and/or results evaluated by control unit 26 can be displayed.

For imaging purposes, corresponding magnetic resonance measurement methods and/or selected pulse sequences are used, which advantageously map the specific magnetic resonance contrast. Conventional T1 weighted and/or T2 weighted sequences, T2*weighted and/or susceptibility-weighted sequences and/or a measurement method, which generates a positive iron contrast, can be used here.

After the magnetic resonance measurement 2 by means of the magnetic resonance tomography apparatus 21, an evaluation 3 of the measured data takes place in the control unit 26. The evaluated results of the magnetic resonance tomography imaging are then shown by way of the screen display 29, with an advantageous imaging with a high contrast of the organ to be examined taking place by using the highly viscous magnetic resonance tomography contrast agent. In particular, an inner wall of a uterus can be displayed with a high contrast ratio in order to identify myoma on the inner wall of the uterus for instance.

1.-13. (canceled)

14. A medical magnetic resonance tomography apparatus for magnetic resonance tomography imaging a cavity in a patient, comprising:
a contrast agent that comprises a highly viscous magnetic resonance contrast agent comprising a substance for generating a specific magnetic resonance contrast.

15. The medical magnetic resonance tomography apparatus as claimed in claim 14, wherein the highly viscous magnetic resonance contrast agent has a viscosity between 2000 mPa·s and 4000 mPa·s.

16. The medical magnetic resonance tomography apparatus as claimed in claim 14, wherein the highly viscous magnetic resonance contrast agent has a pH value between 5.5 and 7.5.

17. The medical magnetic resonance tomography apparatus as claimed in claim 14, wherein the highly viscous magnetic resonance contrast agent has a pH value between 5.5 and 6.0.

18. The medical magnetic resonance tomography apparatus as claimed in claim 14, wherein the highly viscous magnetic resonance contrast agent comprises a buffer solution.

19. The medical magnetic resonance tomography apparatus as claimed in claim 14, wherein the highly viscous magnetic resonance contrast agent comprises at least a highly viscous base material.

20. The medical magnetic resonance tomography apparatus as claimed in claim 14, wherein the substance at least partially comprises a gadolinium chelate.

21. The medical magnetic resonance tomography apparatus as claimed in claim 14, wherein the substance at least partially comprises a fluorine compound.

22. The medical magnetic resonance tomography apparatus as claimed in claim 14, wherein the substance at least partially comprises a hyperpolarized substance.

23. The medical magnetic resonance tomography apparatus as claimed in claim 14, wherein the substance at least partially comprises iron particles.

24. A magnetic resonance contrast agent for medical imaging a cavity in a patient with a magnetic resonance tomography apparatus, comprising:
a highly viscous base material; and
a substance for generating a specific magnetic resonance contrast.

25. The magnetic resonance contrast agent as claimed in claim 24, wherein the magnetic resonance contrast agent has a viscosity between 2000 mPa·s and 4000 mPa·s.

26. The magnetic resonance contrast agent as claimed in claim 24, wherein the magnetic resonance contrast agent has a pH value between 5.5 mPa·s and 7.5 mPa·s.
